(19) United States
(12) Patent Application Publication
(10) Pub. No.: US 2008/0139491 A1
(43) Pub. Date: Jun. 12, 2008

(54) Method For Investigating Cytosine Methylation in Dna Sequences By Means of Triplex-Forming Oligomers

(76) Inventor: Matthias Schuster, Berlin (DE)

Correspondence Address: KRIEGSMAN & KRIEGSMAN, 30 TURNPIKE ROAD, SUITE 9, SOUTHBOROUGH, MA, 01772, US

(21) Appl. No.: 10/562,023

(22) PCT Filed: Jun. 17, 2004
(86) PCT No.: PCT/EP04/06534

§ 371 Date: Dec. 20, 2005

(30) Foreign Application Priority Data
Jun. 20, 2003 (DE) 103 28 813.9

(51) Int. Cl. A61K 31/712; C12Q 1/68; A61P 43/00; C07H 21/00

(52) U.S. Cl. 514/44; 435/6; 536/25.4

(57) ABSTRACT

A method is described for the investigation of cytosine methylation in DNA sequences. Triplex-forming oligomers are utilized, which preferably form triplex structures at positions where cytosine unmethylated at position 5 is present. The triplexes block the transcription, replication and amplification of the DNA. In particular, peptide nucleic acid oligomers with modified nucleobases can be used as triplex-forming oligomers.

The present invention concerns a method for investigating cytosine methylation in DNA sequences. Triplex-forming oligomers are utilized, which preferably form triplex structures at positions where cytosine unmethylated at position 5 is present. The triplexes block the transcription, replication and amplification of the DNA. In particular, peptide nucleic acid oligomers with modified nucleobases can be used as triplex-forming oligomers.

### BACKGROUND OF THE INVENTION

5-Methylcytosine is the most frequent covalently modified base in the DNA of eukaryotic cells. It plays an important biological role, and is involved, among other things, in the regulation of transcription, in genetic imprinting and in tumorigenesis (Millar et al.: Five not four: History and significance of the fifth base. In: The Epigenome, S. Beck and A. Olek, eds.: The Epigenome. Wiley-VCH Publishing Co. Weinheim 2003, pp. 3-20). The identification of 5-methylcytosine as a component of genetic information is thus of considerable interest. It is difficult to detect methylation, of course, since cytosine and 5-methylcytosine have the same base pairing behavior. Many of the conventional detection methods based on hybridization are thus not capable of distinguishing between cytosine and methylcytosine. In addition, the methylation information is completely lost in a PCR amplification.

The usual methods for methylation analysis operate essentially according to two different principles. In the first case, methylation-specific restriction enzymes are utilized, and in the second case, a selective chemical conversion of unmethylated cytosines to uracil is conducted (bisulfite treatment). The enzymatically or chemically pretreated DNA is then amplified for the most part and can be analyzed in different ways (WO 02/072880 pp. 1 ff).

The method according to the present invention permits a direct methylation analysis of the target DNA without prior enzymatic or chemical pretreatment and is thus more rapid and simpler than the conventional methodology. According to the invention, oligomers are utilized, which bind sequence-specifically to the DNA and form triplex structures therein. The binding is preferably conducted at positions at which unmethylated cytosine is present, and this is predominantly caused by steric influences of the methyl group of the 5-methylcytosine. The formation of the triplex and hence the methylation status can be detected in different ways. It is thus possible to detect the unmethylated positions directly by use of labeled triplex-forming oligomers. Methylation can be detected via an amplification of the methylated DNA, while simultaneously, the amplification of unmethylated DNA will be blocked by the triplex structure.

The invention utilizes the knowledge that DNA can be present under certain circumstances in a triplex structure. The triplex formation is promoted by a homopurine-homopyridine sequence in the DNA double strand. A third DNA strand can be deposited sequence-specifically in the large groove of the double strand, whereby this gives rise to the formation of hydrogen bonds with the homopurine sequence. Two different triplex structures occur, depending on the relative orientation of the third strand of the purine strand. In the so-called pyrimidine pattern, the third strand is rich in pyrimidine (Y) and binds parallel to the purine (R) strand of the double helix (Y-RY pattern). In this way, two Hoogsteen hydrogen bonds are formed each time between thymine and adenine (T-AT) as well as between protonated cytosine and guanine (C⁺-GC). The Y-RY triplexes are stable only under acidic conditions. In the purine pattern one purine-rich third strand binds anti-parallel to the purine strand of the duplex (R-RY). Two reverse Hoogsteen base pairings result between guanine and guanine (G-GC), adenine and adenine (A-AT) or thymine and adenine (T-AT). The purine structure is independent of pH and more stable than the pyrimidine pattern. The formation of both types of triplexes is dependent on chain length, base composition, concentration of divalent cations and temperature (Guntaka et al.: Triplex□forming oligonucleotides as modulators of gene expression. Int J Biochem Cell Biol. 2003 January; 35 (1): 22-31).

Since it is necessary for the triplex formation that the third strand interacts with a homopurine sequence in the double strand, basically only GC and AT, and not CG und TA base pairs can be detected in the double strand. The triplex is destabilized by approximately 2.5-4.0 Kcal/mol for each erroneous pairing; the stability of the triplex formation is reduced by a factor of 10-100 or more (Vasquez and Wilson: Triplex-directed modification of genes and gene activity. Trends Biochem Sci. 1998 January; 23 (1): 4-9 1998 pp. 4, 5). Recently there have been various attempts to extend the triplex recognition code to the other two Watson-Crick base pairs. The highly promising combinations are G-TA and T-CG. T-CG can occur both in a parallel structure as well as in an antiparallel structure, whereas G-TA occurs only in the parallel form (Gowers and Fox: Towards mixed sequence recognition by triple helix formation. Nucleic Acids Res. 1999 Apr. 1; 27 (7): 1569-77). Of course, these triplexes are also less stable than the canonical YRY triplexes. One reason for this lies in the fact that in both cases only a single hydrogen bond is formed between T-CG and G-TA, whereas the canonical triads are stabilized by two hydrogen bonds. In addition, the base stacking interactions appear to be destabilized over long distances (Coman and Russu: Site-resolved energetics in DNA triple helices containing G*TA and T*CG triads. Biochemistry. 2002 Apr. 2; 41 (13): 4407-14, with further citations).

In the past, a pyrimidine recognition of the double strand has been successfully achieved by chemical modification of the bases of the triplex strand. For a cytosine recognition within parallel triplexes, N⁴-substituted cytosine derivatives with side chains, which also can form hydrogen bonds to guanine, are particularly suitable (Gowers and Fox 1999, loc. cit., p. 1573 with further citations; Vasquez and Glaser: Triplex-forming oligonucleotides: principles and applications. Q. Rev. Biophys. 2002 February; 35 (1): 89 ff, 98). Particularly suitable are N⁴-(3-acetamidopropyl)cytosine and N⁴-(6 amino-2-pyridinyl)cytosine (FIGS. 1 and 2).

In addition to modified bases, a plurality of modifications of the third strand have been developed in order to stabilize triplexes and reduce their degradation in cells and tissues. Thus, in the case of the triplex-forming oligonucleotides which were used, the phosphorus atoms in the phosphate backbone have been replaced by sulfur, the OH groups in the ribose and the purine rings have been methylated or the 5′ and 3′ ends have been blocked by different components (Guntaka et al. 2003, loc. cit., p. 23 with further citations). Other triplex-forming molecules are also used, particularly peptide nucleic acids (PNA). It is interesting in this regard that very stable triplexes can be formed from triplex-forming PNAs with DNA-PNA duplexes (PNA₂-DNA triplexes; Ray and Norden: Peptide nucleic acid (PNA): its medical and biotechnical applications and promise for the future. FASEB J. 2000 June; 14 (9): 1041 ff, 1048).

The triplex formation is the foundation of several medical and biological applications, e.g., for transcription modulation, for site-directed mutagenesis, for promoting recombination, and for inhibiting polymerases (Guntaka et al. 2003, loc. cit. p. 26 f, Vasquez and Glaser 2002, loc. cit. p. 98 ff, each with further citations).

### DESCRIPTION OF THE INVENTION

The object of the present invention is to provide a novel method for the detection of methylated positions.

The object is solved by the characterizing features of the main claim. Advantageous enhancements of the method according to the invention are characterized in the dependent subclaims.

The object is solved by a method for the detection of cytosine methylations in DNA, wherein the DNA to be investigated is brought into contact with a triplex-forming molecule, which distinguishes between methylated and unmethylated DNA.

According to the invention, it is preferred that the triplex-forming molecule forms a triplex with the DNA to be investigated, whereby the triplex formation is preferred for unmethylated DNA as opposed to triplex formation for methylated DNA, and the triplex formation is used for the detection of the methylation status.

According to the invention, it is also preferred that oligonucleotides, peptide nucleic acid (PNA) oligomers, other oligonucleotide analogs or chimeras, or molecules derived from these substance classes are used as the triplex-forming molecules.

It is further preferred that the triplex-forming molecule bears both a duplex-forming as well as also a triplex-forming sequence.

It is also preferred that the triplex-forming molecule bears at least one modified nucleobase, which specifically or selectively binds to a cytosine in the triplex. It is further preferred that N⁴-substituted cytosine derivatives are used as the nucleobase. It is also preferred here that N⁴-(3-acetamidopropyl)cytosine or N⁴-(6-amino-2-pyridinyl)cytosine is used as the nucleobase. It is most particularly preferred that N⁴-substituted cytosines which bear additional modifications at position 3 are used as the nucleobase. It is particularly preferred also that position 3 is modified with a methyl, ethyl or isopropyl group.

A method in which the triplex-forming molecule bears a detectable label is also preferred according to the invention.

A method is preferred, in which the methylation status is detected via an in-situ hybridization.

In addition, a method is preferred, in which the DNA is amplified for detection of the methylation status, wherein the methylated DNA is preferentially amplified over unmethylated DNA due to the triplex formation.

A method is also particularly preferred, in which the DNA is amplified for detection of the methylation status, wherein unmethylated DNA is preferentially amplified over methylated DNA due to the triplex formation.

It is particularly preferred that triplex-forming molecules are utilized, which also serve as primers in the amplification.

A method is also preferred, in which structures which hinder an amplification are formed by the triplex formation.

It is particularly preferred that deoxy-5-methylcytosine triphosphate, and not deoxycytosine triphosphate (dCTP), is utilized in the amplification.

In addition, it is preferred that a real-time PCR is utilized for the amplification.

According to the invention, a method is also provided for separating methylated and unmethylated DNA, wherein

(a) the DNA is brought into contact with a triplex-forming molecule,
(b) the triplex-forming molecule forms a triplex with the DNA, wherein triplex formation is preferred with unmethylated DNA over triplex formation with methylated DNA, and
(c) the triplex formation is utilized for the separation.

According to the invention, a method is also provided for the specific introduction of DNA damage into unmethylated DNA, wherein

(a) the DNA is brought into contact with a triplex-forming molecule, which bears a reactive chemical group,
(b) the triplex-forming molecule forms a triplex with the DNA, wherein triplex formation with unmethylated DNA is preferred over triplex formation with methylated DNA,
(c) one reacts the reactive chemical group with the DNA present in the triplex form.

In addition, according to the invention, a method is also provided for the specific inhibition of replication of unmethylated DNA, wherein

(a) the DNA is brought into contact with a triplex-forming molecule,
(b) the triplex-forming molecule forms a triplex with the DNA, wherein triplex formation with unmethylated DNA is preferred over triplex formation with methylated DNA,
(c) the replication of the DNA present in the triplex form is inhibited.

According to the invention, a method is also provided for the specific inhibition of the transcription of unmethylated DNA, wherein

(a) the DNA is brought into contact with a triplex-forming molecule,
(b) the triplex-forming molecule forms a triplex with the DNA, wherein triplex formation with unmethylated DNA is preferred over triplex formation with methylated DNA,
(c) the transcription of the DNA present in the triplex form is inhibited.

Another subject of the present invention is the use of oligonucleotides, peptide nucleic acid (PNA) oligomers, other oligonucleotide analogs or chimeras, or molecules derived from these classes of substances, which contain N⁴-(3-acetamidopropyl)cytosine, N⁴-(6-amino-2-pyridinyl)cytosine or other N⁴-substituted cytosine derivatives, for the therapy of disorders which are associated with demethylation of cytosines.

The method according to the invention for distinguishing between methylcytosine and cytosine utilizes steric hindrance, which proceeds from the methyl group of the methylcytosine and which can prevent the binding of specific triplex-forming oligomers. In addition to steric considerations, electronic influences of the methyl group probably also play a role. Both oligonucleotides and peptide nucleic acid (PNA) oligomers can be utilized as triplex-forming oligomers. The use of other oligonucleotide analogs or chimeric molecules derived from the above-named classes of substances is also possible. The binding code for the third strand and the preferred conditions under which a triplex formation occurs are prior art (see above; see also the citations in U.S. Pat. No. 6,461,810 B1, column 3, lines 30ff). For cytosine detection, it is considered that basically an interaction of the third strand with a homopurine sequence is necessary for the triplex formation, but cytosine is a pyrimidine base. Therefore, the use of a modified base is necessary for cytosine detection. N⁴-substituted cytosine derivatives with side chains that also form hydrogen bonds to guanine are particularly suitable within parallel triplexes and thus increase the stability of the triplex. For distinguishing between cytosine and methylcytosine, it is necessary that the modified bases are structured in such a way that a triplex formation is made difficult sterically by the 5-methyl group of the cytosine. N⁴-(3-acetamidopropyl)cytosine and N⁴-(6-amino-2-pyridinyl)cytosine, e.g., are particularly preferred as modified bases. The production of these bases is prior art (Gowers and Fox 1999, loc. cit., p. 1573 with further citations). It is to be expected that the steric hindrance and thus the capacity for distinguishing between cytosine and methylcytosine is further increased, if the utilized N⁴-substituted cytosine derivatives bear additional modifications at position 3, e.g., methyl, ethyl or isopropyl substituents.

A plurality of other modifications of the sugars and backbone of the nucleotides, which can be utilized in order to stabilize the triplex, also belong to the prior art. In particular, peptide nucleic acid (PNA) oligomers are used (Guntaka et al. 2003, loc. cit., with further citations). It is also known that the triplex formation can be stabilized by intercalators or triplex-specific ligands (Sun: New targets for triple helix forming oligonucleotides. pp. 273 ff, 276; Escude and Garestier: Triple helix stabilizing agents pp. 257 ff, each found in: C. Malvy, A. Harel-Bellan, L L Pritchard, eds: Triple helix-forming oligonucleotides. Kluwer Academic Publishers 1999).

The DNA to be investigated can be present in single as well as double strands. In double-stranded DNA, oligonucleotides are preferably utilized as triplex-forming molecules. If one starts with single-stranded DNA, then first, a duplex formation must precede the triplex formation. Both steps can be mediated by the same molecule, if this molecule bears a sequence that is complementary to the target DNA and also provides a triplex-forming domain. It is particularly preferred here to utilize PNA molecules (“bis-PNA”), since PNA₂-DNA-triplexes are particularly stable (Ray and Norden 2000, loc. cit. p. 1048). The orientation of the Watson-Crick PNA strand is thus antiparallel to the DNA, while the orientation of the Hoogsteen strand is parallel to the DNA. Both sequences are coupled together via a flexible linker. Information on the type and length of the linker are described, e.g., in U.S. Pat. No. 5,693,773 (column 7, lines 64 ff). For the formation of parallel triplexes, a protonation of the cytosines in the third strand is necessary. This pH dependence can be circumvented, however, if the cytosines are replaced by pseudoisocytosines (J). The triplex-forming PNA molecules then contain cytosine in the Watson-Crick sequence and pseudoisocytosine in the Hoogsteen sequence. Bis-PNA molecules can also be utilized for the investigation of double-stranded DNA. Thus the duplex-forming strand of the PNA displaces the corresponding DNA double strand (see: Bentin and Nielsen: Triplexes involving PNA, in: C. Malvy, A. Harel□Bellan, L L Pritchard, eds: Triple helix-forming oligonucleotides. Kluwer Academic Publishers 1999, pp. 245 ff with further citations).

The methylation-dependent triplex formation can be detected in different ways. According to the invention, the triplex-forming molecule is to be labeled and then the triplex, thus the unmethylated state, is to be detected. A preferred embodiment of this method is in-situ hybridization. Unmethylated double-stranded DNA can be detected, e.g., in cells in this way. The sequence specificity of the method according to the invention is thus a particular advantage when compared with other known methods for the in-situ detection of cytosine methylations, e.g., via methylation-specific antibodies.

In particular, chemically modified oligonucleotides can serve as triplex-forming molecules (see above). The oligonucleotides are between 7 and 50 nucleotides, preferably between 10 and 30 nucleotides long. They bear reporter molecules, which can be detected with chemical or physical methods, e.g., biotin, fluorescence or radioactive labels. The exact conditions for an in-situ hybridization with triplex formation are prior art (U.S. Pat. No. 6,461,810 B1, particularly columns 3 ff).

In another variant according to the invention, the detection of methylation is made via an amplification of the methylated DNA, while simultaneously the amplification of unmethylated DNA is blocked by the triplex structure. Current methods, e.g., PCR, can be employed for the amplification. The binding of primers is independent of the methylation status, but the extension of the primers is blocked by the triplex formation at specific sites. It is known that DNA polymerases are not able to decompose triplex structures. The polymerization thus comes to a halt at these sites (WO 96/18732, particularly page 18, lines 17 ff). Due to the particular stability of the triplex, it is preferred to use the above-mentioned bis-PNA molecules, which bear both duplex-forming as well as the triplex-forming sequences, for such blocking. Of course, two different molecules or oligonucleotides can also be used. It is also conceivable and preferred to use other oligonucleotide analogs, e.g., LNA (Locked Nucleic Acids). Methods are known to the person skilled in the art for the production of corresponding oligonucleotide analogs (Braasch and Corey: Locked nucleic acid (LNA): fine-tuning the recognition of DNA and RNA. Chem Biol 2001 January; 8(1):1-7 with further citations).

The concentration of the blocker molecules must be high enough so that a complete blocking is ensured. A concentration range between 100-1000 nM is preferred. The amplification of the methylated DNA is otherwise performed according to the prior art. Care is to be taken, of course, that only 5-methylcytosine nucleoside triphosphates should be utilized. If cytosine were incorporated in the newly synthesized DNA strands, then the triplex-forming molecules would not only bind to the originally unmethylated DNA, but also to the new molecules produced from the originally methylated DNA. The amplificates can be detected in different ways known to the person skilled in the art, e.g., by methods of length measurement such as gel electrophoresis, capillary electrophoresis and chromatography (e.g., HPLC). Also real-time variants can be utilized, e.g., the Taqman or the Lightcycler method.

Another embodiment according to the invention for the selective amplification of methylated DNA lies in the use of primers which simultaneously provide a triplex-forming domain. Oligonucleotides are preferably used for this purpose. The Watson-Crick-forming sequence here lies at the 3′-end of the primer, and the Hoogsteen-forming sequence lies at the 5′-end. As described above, both parts are joined via a linker. The primers are between 30 and 80 nucleotides long, and they are preferably more than 40-60 nucleotides. Amplification can only occur if the primer does not form a triplex, thus if the corresponding cytosine position is methylated. Again, care must be taken to use methylated deoxycytosine triphosphate in the amplification. The amplificates can be detected as described above. According to the invention, the opposite variant is also provided, in which the primers are constructed in such a way that an extension will only be produced if a triplex is formed when the cytosine position is in the unmethylated form.

In addition to the detection of methylated DNA, the method according to the invention can also be used in order to separate methylated sequences from unmethylated ones. Thus, if (a) the DNA is brought into contact with a triplex-forming molecule,

(b) the triplex-forming molecule forms a triplex with the DNA, wherein triplex formation with unmethylated DNA is preferred over triplex formation with methylated DNA, and
(c) the triplex formation is utilized for the separation.

A preferred possibility for this procedure is the triple helix affinity chromatography described in the literature (see: Kamenetskii: Triplexes and biotechnology. In: C. Malvy, A. Harel-Bellan, L L Pritchard, eds: Triple helix-forming oligonucleotides. Kluwer Academic Publishers 1999, 285, 287 ff with further citations).

The methylation-specific triplex formation can also be utilized for further applications. One possibility according to the invention is the sequence-specific introduction of DNA damage into unmethylated DNA. Here,

(a) the DNA is brought into contact with a triplex-forming molecule, which bears a reactive chemical group,
(b) the triplex-forming molecule forms a triplex with the DNA, wherein triplex formation with unmethylated DNA is preferred over triplex formation with methylated DNA,
(c) the reactive chemical group reacts with the DNA pre-sent in triplex form.

Psoralens or alkylating reagents are particularly preferred as reactive chemical groups. The precise conditions of comparable methods are described in the literature (Faria and Giovannangeli, Triplex-forming molecules: from concepts to applications. J Gene Med. 2001 July-August; 3(4):299-310 with further citations).

Other applications of the invention are transcription modulation, replication inhibition, site-directed mutagenesis and promoting the recombination of specific unmethylated DNA sequences. Here,

(a) DNA is brought into contact with a triplex-forming molecule,
(b) the triplex-forming molecule forms a triplex with the DNA, wherein triplex formation with unmethylated DNA is preferred over triplex formation with methylated DNA, and
(c) the replication or the transcription of the DNA pre-sent in triplex form is inhibited, or mutations or recombinations are promoted.

The precise conditions of comparable methods are described in the literature (Faria and Giovannangeli, Triplex-forming molecules: from concepts to applications. J Gene Med. 2001 July-August; 3(4):299-310 with further citations).

According to the invention, the above-named methods can also be used therapeutically. It is known that many disorders are associated with cytosine demethylation of promoter regions or other regulatory regions of specific genes. There is an activation of transcription due to this demethylation. The application of triplex-forming molecules permits a sequence-specific inhibition of transcription or replication or a targeted damage of the corresponding sequences. In particular, oligonucleotides, peptide nucleic acid (PNA) oligomers, other oligonucleotide analogs or chimeras, or molecules derived from these classes of substances, are considered as possible therapeutic agents, which contain N⁴-substituted cytosine derivatives, e.g., N⁴-(3-acetamidopropyl)cytosine or N⁴-(6-amino-2-pyridinyl)cytosine. The oligomers can be administered together with a pharmaceutical vehicle and possibly via different pathways with additional adjuvants. It is also possible to combine them with other therapeutic agents. The precise composition and the type of administration can be determined by the person skilled in the art according to conventional pharmaceutical principles.

### BRIEF DESCRIPTION OF THE FIGURES

FIG. 1 shows N⁴-(3-acetamidopropyl)cytosine and the formation of hydrogen bonds with a C-G pair. The figure is taken from Gowers and Fox 1999, loc. cit.

FIG. 2 shows N⁴-(6-amino-2-pyridinyl)cytosine and the formation of hydrogen bonds with a C-G-pair. The figure is taken from Gowers and Fox 1999, loc. cit.

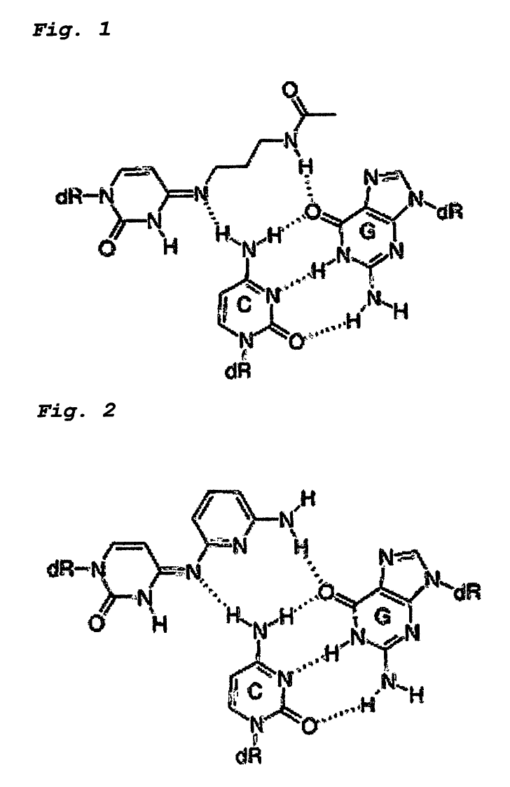

### EXAMPLES

### Example 1

### Detection of Cytosine Methylations by Use of bis-PNA Molecules

The underscored cytosines presented in the following DNA sequence will be detected in their methylated state, but not in their unmethylated state. Detection is made by PCR, in which the presented sequence is amplified in the presence of this methylation, but not in its absence.

(SEQ ID: 1)

5′-CGGAGGAAGAAAGAGGAGGGGCTGGCTGGTCACCAGAGGGTGGGGC
G-GACCGCGTGCGCTCGGCGGCTGCGGAGAGGGGGAGAGCAGG-CAGCGG
GCGGCGGGGAGCAGCATGGAGCCGGCGGCGGGGAGCAGCATGGAGCCTT-
3′

For this purpose, isolated and purified DNA is subjected to PCR, with the use of the primers CGGAGGAAGA-AAGAGGAG and AAGGCTCCATGCTGCTC (both at concentrations of 300 nM each), in the presence of 100 nM of the bis-PNA molecule NH2-CTCCCCGCCGC-0-0-0-JXJJXJJJJTJ-COOH (X═N⁴-(3-acetamidopropyl)cytosine; 0=8-amino-3,6-dioxaoctanoic acid; J=pseudoisocytosine) in a reaction volume of 0.02 ml (55° C. annealing temperature, 72° C. primer extension temperature, 95° C. denaturing temperature, 42 reaction cycles). Deoxy-5-methylcytosine triphosphate is utilized instead of deoxycytosine triphosphate. The amplificate with a length of 143 base pairs is detected after separation of the PCR product by agarose gel electrophoresis in the presence of ethidium bromide by visualization in ultraviolet light.

The modified cytosine is synthesized as described in the literature (Gowers and Fox 1999, loc. cit., p. 1573 with further citations). The synthesis of the PNA molecules is also prior art (see also the citations in Ray and Norden 2000, loc. cit., p. 1042).

### Example 2

### Detection of Cytosine Methylations with the Use of PNA-DNA Hybrid Molecules

The underscored cytosines presented in the following DNA sequence will be detected in their methylated state, but not in their unmethylated state. Detection is made by PCR, in which the presented sequence is amplified in the presence of methylation, but not in its absence. For this purpose, a PCR primer with a PNA domain is modified in such a way that when methylation is absent, but not when it is present, a triplex structure is formed, which pre-vents the binding of the DNA polymerase and thus the amplification.

(SEQ. ID: 2)

5′-CGGAGGAAGAAAGAGGAGGGGCTGGCTGGTCACCAGAGGGTGGGG-C
GGACCGCGTGCGCTCGGCGGCTGCGGAGAGGGGGAGAGCAGG-CAGCGGG
CGGCGGGGAGCAGCATGGAGCCGGCGGCGGGGAGCA-3′

Isolated and purified DNA is subjected to PCR with the use of the primer CGGAGGAAGAAA-GAGGAG and the PNA-DNA hybrid molecule (both 300 nM) NH2-XJJXJJXJJJJTJ-O-O-O-O-(T)GCTCCCCGCCGCCG-3′(DNA monomers in italics; X═N⁴-(3-acetamidopropyl)cytosine; 0=8-amino-3,6-dioxaoctanoic acid; J=pseudoisocytosine; (T)=5′-aminothymidine) in a reaction volume of 0.02 ml (55° C. annealing temperature, 72° C. primer extension temperature, 95° C. denaturing temperature, 42 reaction cycles). Deoxy-5-methylcytosine triphosphate is utilized as the cytosine deoxynucleoside triphosphate. The amplificate with a length of 143 base pairs is detected after separation of the PCR product by agarose gel electrophoresis in the presence of ethidium bromide by visualization in ultraviolet light.

The synthesis of PNA-DNA hybrid molecules is prior art (see, e.g., Uhlmann et al. Angew. Chem. 1998, 110, 2954-83 and the citations contained therein).

### Example 3

### Methylation-Specific Labeling of DNA by Use of Biotinylated bis-PNA Molecules

The following sequence will be labeled with a biotin group in the region of the two underscored cytosines via the binding of a bis-PNA only if these cytosines are pre-sent in their unmethylated state, but not if they are in their methylated form.

(SEQ ID 3)

5′-CGGAGGAAGAAAGAGGAGGGGCTGGCTGGTCACCAGAGGGTGGGGC
G-GACCGCGTGCGCTCGGCGGCTGCGGAGAGGGGGAGAGCAGG-CAGCGG
GCGGCGGGGAGCAGCATGGAGCCGGCGGCGGGGAGCAGCATGGAGCCTT-
3′

For this purpose, isolated and purified DNA is dissolved in 100 mM phosphate buffer (pH 7-8, 500 mM NaCl) containing 100 nM biotinyl-NH-CTCCCCGCCGC-0-0-0-JXJJXJJJJTJ-COOH (X═N⁴-(3-acetamidopropyl)cytosine; 0=8-amino-3,6-dioxaoctanoic acid; J=pseudoisocytosine) and, after a ten-minute denaturing at 95° C., is incubated for three hours at 55° C. After separation of the excess bis-PNA, for example, by ultrafiltration or gel filtration, the labeled DNA is available for further investigations.

1. A method for the detection of cytosine methylation in DNA characterized in that the DNA to be investigated is brought into contact with a triplex-forming molecule which distinguishes between methylated and unmethylated DNA.

2. The method according to claim 1, further characterized in that the triplex-forming molecule forms a triplex with the DNA to be investigated, whereby triplex formation with unmethylated DNA is preferred over triplex formation with methylated DNA, and the triplex formation is used for the detection of the methylation status.

3. The method according to claim 1, further characterized in that oligonucleotides, peptide nucleic acid (PNA) oligomers, other oligonucleotide analogs or chimeras, or molecules derived from these classes of substance are used as the triplex-forming molecules.

4. The method according to claim 1, further characterized in that the triplex-forming molecule consists both a duplex-forming sequence as well as a triplex-forming sequence.

5. The method according to claim 1, further characterized in that the triplex-forming molecule comprises at least one modified nucleobase, which specifically or selectively binds to a cytosine in the triplex.

6. The method according to claim 5, further characterized in that N⁴-substituted cytosine derivatives are used as nucleobases.

7. The method according to claim 5, further characterized in that N⁴-(3-acetamidopropyl)cytosine or N⁴-(6-amino-2-pyridinyl)cytosine is used as the nucleobase.

8. The method according to claim 5, further characterized in that N⁴-substituted cytosines, which comprise additional modifications at position 3, are used as nucleobases.

9. The method according to claim 8, further characterized in that position 3 is modified with a methyl, ethyl or isopropyl group.

10. The method according to claim 1, further characterized in that the triplex-forming molecule bears a detectable label.

11. The method according to claim 1, further characterized in that the methylation status is detected via an in-situ hybridization.

12. The method according to claim 1, further characterized in that for the detection of the methylation status, the DNA is amplified, wherein due to the triplex formation, the amplification of methylated DNA is preferred over the amplification of unmethylated DNA.

13. The method according to claim 1, further characterized in that for the detection of the methylation status, the DNA is amplified, wherein due to the triplex formation, the amplification of unmethylated DNA is preferred over the amplification of methylated DNA.

14. The method according to claim 12, further characterized in that triplex-forming molecules are utilized, which also serve as primers in the amplification.

15. The method according to claim 1, further characterized in that structures which hinder amplification are formed by the triplex formation.

16. The method according to claim 12, further characterized in that deoxy-5-methylcytosine triphosphate, and not deoxycytosine triphosphate (dCTP), is utilized in the amplification.

17. The method according to claim 12, further characterized in that a real-time PCR is utilized for the amplification.

18. A method for the separation of methylated and unmethylated DNA characterized in that
(a) the DNA is brought into contact with a triplex-forming molecule,
(b) the triplex-forming molecule forms a triplex with the DNA, wherein triplex formation with unmethylated DNA is preferred over triplex formation with methylated DNA,
(c) the triplex formation is utilized for the separation.

19. A method for the specific introduction of DNA damage into unmethylated DNA, characterized in that
(a) the DNA is brought into contact with a triplex-forming molecule which bears a reactive chemical group,
(b) the triplex-forming molecule forms a triplex with the DNA, wherein triplex formation with unmethylated DNA is preferred over triplex formation with methylated DNA,
(c) the reactive chemical group is reacted with the DNA present in triplex form.

20. A method for the specific inhibition of replication of unmethylated DNA, characterized in that
(a) DNA is brought into contact with a triplex-forming molecule,
(b) the triplex-forming molecule forms a triplex with the DNA, wherein triplex formation with unmethylated DNA is preferred over triplex formation with methylated DNA,
(c) the replication of the DNA present in triplex form is inhibited.

21. A method for the specific inhibition of transcription of unmethylated DNA, characterized in that
(a) DNA is brought into contact with a triplex-forming molecule,
(b) the triplex-forming molecule forms a triplex with the DNA, wherein triplex formation with unmethylated DNA is preferred over triplex formation with methylated DNA,
(c) the transcription of the DNA present in triplex form is inhibited.

22. Use of oligonucleotides, peptide nucleic acid (PNA) oligomers, other oligonucleotide analogs or chimeras, or molecules derived from these substance classes, which contain N⁴-(3-acetamidopropyl)cytosine, N⁴-(6-amino-2-pyridinyl)cytosine or other N⁴-substituted cytosine derivatives, for the therapy of disorders which are associated with cytosine demethylation.


SEQUENCE LISTING (3 sequences)

SEQ ID NO: 1 - DNA, 143 bp, HOMO SAPIENS
cggaggaagaaagaggaggggctggctggtcaccagagggtggggcggaccgcgtgcgctcggcggctgcggagagggggagagcaggcagcgggcggcggggagcagcatggagccggcggcggggagcagcatggagcctt

SEQ ID NO: 2 - DNA, 131 bp, HOMO SAPIENS
cggaggaagaaagaggaggggctggctggtcaccagagggtggggcggaccgcgtgcgctcggcggctgcggagagggggagagcaggcagcgggcggcggggagcagcatggagccggcggcggggagca

SEQ ID NO: 3 - DNA, 143 bp, HOMO SAPIENS; identical to SEQ ID NO: 1